SEQUENCE LISTING (3 sequences)

SEQ ID NO: 1 - DNA, 28 bp, Artificial
  Synthetic Oligonucleotide; Termed Oligo A when biotinylated at 5′ position and  Cy3 labeled at 3′ position; Termed Oligo B when biotinylated at 5′ position and not labeled at 3′ position
aatccagatatagtcatctagcaataca

SEQ ID NO: 2 - DNA, 36 bp, Artificial
  Synthetic oligonucleotide; Termed Oligo C when Cy3 labeled at 3′ position; Termed Oligo E when not labeled at 3′ position
ttagctcgactcagggatccggattgtattgctaga

SEQ ID NO: 3 - DNA, 12 bp, Artificial
  Synthetic oligonucleotide; Termed Oligo F when Cy3 labeled at 3′ position
atccggatccct

(19) United States
(12) Patent Application Publication
(10) Pub. No.: US 2005/0221283 A1
(43) Pub. Date: Oct. 6, 2005

(54) Biochip

(76) Inventors: Vijay K. Mahant, Murrieta, CA (US); Fareed Kureshy, Del Mar, CA (US)

Correspondence Address: Robert D Fish;Rutan & Tucker, 14th Floor, 611 Anton Boulevard, Costa Mesa, CA, 92626, US

(21) Appl. No.: 10/433,766

(22) PCT Filed: Dec. 11, 2001
(86) PCT No.: PCT/US01/47991

§ 371 Date: Oct. 10, 2003

(51) Int. Cl. C12Q 1/70; G01N 33/53; C12M 1/34

(52) U.S. Cl. 435/5; 435/7.1; 435/287.2

(57) ABSTRACT

Improved biochips comprise a matrix layer coupled to a substrate, wherein the matrix layer includes a plurality of ligands in a plurality of predetermined positions and wherein ligands bind to an anti-ligand disposed in a sample fluid. Preferred matrix layers are multi-functional matrix layers that reduce autofluorescence, incident-light-absorption, charge-effects, and/or surface unevenness of the substrate, and contemplated biochips may comprise additional matrix layers. Contemplated biochips may be useful in detection and/or quantification of various anti-ligands, including polypeptides, polynucleotides, carbohydrates, pharmacologically active molecules, bacterial or eukaryotic cells, and/or viruses.

This application claims the benefit of U.S. patent application Ser. No. 09/735,402 filed Dec. 12, 2000, which is incorporated herein by reference.

### FIELD OF THE INVENTION

The field of the invention is analytical devices.

### BACKGROUND OF THE INVENTION

Genomics and proteomics research made a vast number of nucleotide and peptide sequences available for analysis. Consequently, high-throughput screening of samples for the presence and/or quantity of a vast number of known genes or polypeptides has gained considerable interest in recent years. There are various devices and methods known in the art, and many of those devices and methods are adapted for screening of multiple nucleic acid sequences using immobilized nucleic acid probes. Among other applications, the use of such probes in a microarray allows massive parallel experiments in various fields, including pharmacogenomics, gene expression, compound screening, toxicology, single nucleotide polymorphism (SNPs) analysis, and short tandem repeats (STRs) analysis.

While there are numerous methods of immobilization of nucleic acid sequences to a carrier are known in the art, all or almost all of them suffer from one or more disadvantage. For example, in a southern- or northern blot type system, the nucleic acid to be analyzed is blotted and immobilized (e.g., via UV irradiation) onto a solid phase (e.g., nylon or nitrocellulose membrane) and after blocking of non-specific binding sites probed with one or more probes having complementary sequence to the sequence of interest. While such immobilization is relatively simple, substrates (e.g., glass, membranes) in such assays exhibit often relatively high intrinsic background signals. Furthermore, membranes employed in such systems often require elaborate and/or time-consuming blocking steps. Furthermore, depending on the detection system (e.g., radioisotope detection, fluorescence, chemiluminescence detection, etc.) direct detection of the hybridized probe is frequently difficult, and often relatively insensitive. Moreover, high-density loading of membranes is often difficult to achieve due to the relatively hydrophobic character of many of such membranes.

To circumvent at least some of the problems associated with relatively low anti-ligand or ligand density on a solid support, various approaches have been performed. In one approach, as described in U.S. Pat. No. 5,279,951 to Pegg, et al. nucleic acids or other probe molecules are immobilized to a plastic substrate using a heterobifunctional cross-linker in which a central ring structure having a hydrophobic hydrocarbon chain binds to the plastic substrate, and in which at least one hydrophilic chain with a terminal reactive group reacts with the probe molecule. While such heterobifunctional cross-linkers generally allow a relatively high ligand or anti-ligand density, the covalent bond formation between the heterobifunctional cross-linker and the probe molecule (i.e., ligand or anti-ligand ) is typically not specific to a particular epitope or particular reactive group in a plurality of reactive groups. Moreover, suitable plastic substrates need to have a relatively hydrophobic surface to effectively bind the heterobifunctional cross-linker, which may be problematic if the volume of the sample fluid applied to the substrate is relatively small.

In another approach, probe molecules are attached to a solid support without a heterobifunctional cross-linker as described in U.S. Pat. No. 5,262,297 to Sutton et al. Sutton's solid polymer support includes a copolymers which contain a plurality of reactive carboxylic acid groups that are extended from the polymer support surface with a linking group having from 8 to 50 atoms and two or more alkylene, arylene, alkylenearylene or arylenealkylene groups. Probe molecules are then reacted with the reactive groups to attach the probe molecule to the solid polymer support. However, depending on the particular chemical nature of the probe molecule, reaction of the reactive group and the probe molecule may require reaction conditions that are detrimental to the chemical or conformational stability of the probe molecule. Furthermore, unreacted reactive groups need to be blocked prior to analysis, thereby significantly increasing assay time and reagent costs.

Alternatively, as described in U.S. Pat. No. 5,034,428 to Hoffman et al. probe molecules are immobilized (i.e., grafted) onto a hydrophilic polymeric substrate which has been pre-irradiated with ionizing radiation. The pre-irradiation step is typically carried out at −78° C. in air, while the grafting step is carried out at 0° C. in an oxygen free atmosphere. While Hoffman's grafting technique provides for covalent attachment of the probe molecules and may even be employed at relatively high probe molecule density, various difficulties remain, including chemical or conformational stability of the probe molecule, and relatively high equipment cost.

In a further approach, nucleic acid test arrays are produced using a photolithographic process, thereby allowing relatively high density of capture probes (e.g., greater than 10000 probes per array). Systems for such high-density arrays are described, for example, in U.S. Pat. Nos. 5,599,695, 5,843,655, and 5,631,734. While high-density arrays are particularly useful for sequencing or complex genetic analysis, numerous disadvantages remain. For example, custom synthesis of such high-density arrays is likely cost-prohibitive for all but a few individuals and/or organizations. Moreover, due to the particular chemistry employed in building such arrays, non-nucleic acid probes (e.g., receptors, antibodies, and other polypeptides) are difficult, if at all, to implement. Alternatively, solid phase nucleic acid synthesis may be performed as described by Maskos and Southern, (Nucleic Acid Research 1679. 1992), in which a linker system is employed for the attachment of a nucleic acid to a glass support. However, similar problems as described above remain.

Moreover, all or almost all of the known systems require a substantially planar surface of the substrate to which the probe molecule is attached to, and especially in systems that rely on optical detection of a bound anti-ligand. Non-planar surfaces in such systems typically generate false-negative or significantly reduced test results for at least some of the probe molecules attached to the surface. Still further, optical detection may further be complicated in many of the known systems where the support is optically active (i.e., absorbs or reflects incident light, exhibits autofluorescence, etc.).

Thus, although various systems for attachment of probe molecules to a biochip are known in the art, numerous problems still remain. Therefore, there is still a need for an improved methods and systems for attachment of probe molecules to a biochip.

### SUMMARY OF THE INVENTION

The present invention is directed to an improved biochip in which a matrix layer is coupled to a substrate and further includes a plurality of ligands that specifically bind to one or more anti-ligands disposed in a sample fluid. Preferred substrates include organic polymers or inorganic polymers or materials (e.g., polyethylene, polyester, polystyrene, glass, or metal), while preferred matrix layers include an aqueous solvent and a gel (e.g., agarose, polyacrylamide, or gelatin). Contemplated matrix layers may be coupled to the substrates via a hydrophilic interposed layer.

In one aspect of the inventive subject matter, the matrix layer is a multi-functional matrix layer that provides reduction of autofluorescence, incident-light-absorption, charge-effects, and/or surface unevenness of the substrate. Further contemplated biochips may include additional matrix layers, wherein at least one of the matrix layers may comprise a surfactant, a humectant, a buffer, and/or a light-absorbing agent.

In another aspect of the inventive subject matter, contemplated ligands include a nucleotide, a polypeptide, a polynucleotide, a carbohydrate, a pharmacologically active molecule, a bacterial cell, a eukaryotic cell, and/or a virus (or fragments thereof). Consequently, contemplated anti-ligands may include a nucleotide, a polypeptide, a polynucleotide; a carbohydrate, a pharmacologically active molecule, a bacterial cell, a eukaryotic cell, and/or a virus (or fragments thereof), all of which may be disposed in a sample fluid (e.g., being or derived from blood, serum, plasma, urine, spinal fluid, sputum, buffer, cell lysate, etc.).

In a further aspect of the inventive subject matter, the ligand is at least partially embedded within the multi-functional matrix layer. Alternatively, contemplated ligands may be coupled to the multi-functional matrix layer via a coupling moiety, wherein suitable coupling moieties may comprise a first portion (e.g., avidin or streptavidin) that is coupled to the matrix layer and a second portion (e.g., biotin) that is coupled to the ligand, wherein the first and second portions form a non-covalent bond with each other.

In a still further aspect of the inventive subject matter, contemplated biochips may include a first plurality of ligands and a second plurality of ligands, wherein first and second plurality of ligands belong to distinct classes (e.g., a polypeptide, a polynucleotide, a carbohydrate, a pharmacologically active molecule, a bacterial cell, an eukaryotic cell, and a virus).

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a schematic horizontal cross sectional view of an exemplary biochip according to the inventive subject matter.

FIG. 2 is a schematic top view of another exemplary biochip according to the inventive subject matter.

FIG. 3 is a schematic horizontal cross sectional view of a further exemplary biochip according to the inventive subject matter.

### DETAILED DESCRIPTION

As used herein, the term “biochip” refers to a support onto which a plurality of ligands is coupled at a plurality of predetermined positions. While the number of ligands in contemplated biochips is not critical to the inventive subject matter, it is generally preferred that suitable biochips include at least 10, more preferably at least 100, and most preferably at least 1000 chemically or conformationally distinct ligands.

As further used herein, the term “ligand” generally refers to any molecule, complex of molecules, or cell that binds to an anti-ligand with a dissociation constant K_{D}≦10⁻²M, and more typically K_{D}≦10⁻³M, at a temperature of 25° C. and physiological buffer conditions (e.g., pH between 6.5 and 8.5, and ionic strength sufficient to maintain native conformation, viability, and/or Watson-Crick hybridization (between ligand and anti-ligand) of the anti-ligand). The term “anti-ligand” as used herein refers to any molecule, complex of molecules, or cell that binds to the ligand with a dissociation constant of K_{D}≦10 ⁻²M and more typically K_{D}≦10⁻³M, at a temperature of 25° C. and physiological buffer conditions (ie., pH between 6.5 and 8.5, and ionic strength sufficient to maintain native conformation, viability, and/or Watson-Crick hybridization (between ligand and anti-ligand) of the anti-ligand). Thus, it should be recognized that a ligand and an anti-ligand form an affinity pair.

As still further used herein the term “matrix” refers to a substantially non-fluid supporting medium in which a substance (i.e., a ligand, an additive, and/or a coupling agent) is at least partially embedded, and wherein the substance may further be covalently bound to the supporting medium. The term “substantially non-fluid supporting medium” as used herein means that no more than 10 vol % of the supporting medium will move over a period of 60 minutes at 25° C. from a surface having the same size as the supporting medium when the surface (onto which the supporting medium is directly attached) is positioned from a horizontal to a vertical position.

Contemplated supporting media may be chemically homogeneous (ie., consist of only one molecular species) or heterogeneous (i.e., consists of more than one molecular species). For example, suitable supporting media include gels (e.g., gelatin, agarose, polyacrylamide, etc.), porous three-dimensional polymeric networks (e.g., polyarylene ethers, silicon-containing inorganic polymers), semi-interpenetrating or interpenetrating polymeric networks, and so forth. In contrast, a fluid colloid or a solvent are not considered a substantially non-fluid supporting medium under the scope of this definition. Furthermore, a multi-functional (e.g., homo- or heterobifunctional, or higher) crosslinker or a layer thereof is not considered a matrix.

It is generally contemplated that improved biochips comprise a matrix layer that is coupled to a substrate and further includes a plurality of ligands in a plurality of predetermined positions, wherein the ligands bind to one or more anti-ligands disposed in a sample fluid when the sample fluid contacts at least a portion of the matrix.

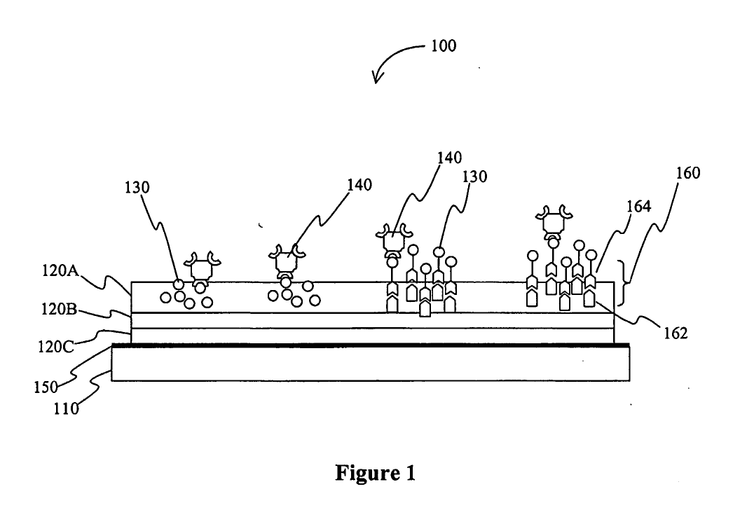

More particularly, as shown in FIG. 1, an exemplary biochip 100 includes a substrate 110 onto which via a hydrophilic interposed layer 150 a matrix layer or a plurality of matrix layers 120A, 120B, and 120C are disposed. Embedded within the matrix layer 120A is a plurality of ligands 130 in predetermined positions. Some of the ligands 130 bind anti-ligand 140. Also (and at least partially) embedded in the matrix layer 120A is a coupling moiety 160 that comprises a first portion 162 and a second portion 164, wherein the second portion is coupled to ligand 130, and wherein some of the ligands 130 that are coupled to the coupling moiety 160 bind anti-ligand 140.

With respect to the substrate it is generally preferred that contemplated substrates will have a generally sheet-like configuration with a length and width of about 2.0-3.0 cm and a thickness of about 0.1 mm-1.0 mm. Preferred substrates further comprise an organic or inorganic polymer, and especially preferred organic polymers include polyethylene, polystyrene, or a polyester (most preferably Mylar® (polyethylene phthalate polymer)). However, in alternative aspects of the inventive subject matter, the configuration and material of suitable substrates may vary considerably. For example, where relatively stiff materials (i.e., materials that will not deform when manual force is applied) are preferred, contemplated materials include metals (e.g., aluminum), metal alloys (e.g., brass), or glass (e.g., borosilicate glass). Among other things, stiff materials are contemplated to be advantageous where the substrate will form an integral part of a housing for contemplated biochips. On the other hand, where relatively flexible materials are preferred (i.e., materials that will deform when manual force is applied), suitable materials will especially include organic polymeric films (e.g., polyethylene, polystyrene, or a polyester, nitrocellulose, paper, etc.). Furthermore, appropriate materials may also be selected according to one or more of alternative parameters, including thermal properties (e.g., heat transfer), mechanical properties (e.g., resilience to ultrasound), optical properties (e.g., reflectivity, absorption, or autofluorescence). Moreover, contemplated substrates may also include combinations of two or more of the materials indicated above.

Similarly, contemplated substrates may any suitable size or shape, and it should be recognized that all sizes and shapes are appropriate so long as such sizes and shapes will at least partially support a matrix layer that is coupled to the substrate. For example, where the ligand density is relatively low, sizes of 2.0-10.0 cm (width and/or length) and even larger are contemplated. In contrast, and especially where the number of ligands is relatively low (e.g., at or below 100) and the density is relatively high, suitable sizes may be between 1.0-2.0 cm (width and/or length) and even smaller. With respect to the thickness it is contemplated that numerous thickness are suitable, and that a particular thickness will at least in part depend on a particularly desired physical or mechanical property. For example, where the substrate is attached to a carrier, a relatively thin substrate (e.g., between about 0.01 mm-2 mm) may be suitable. In another example, where thermal conductivity and moderate mechanical stability is desired, the thickness may be between 0.5 mm and 5 mm. In a still further example, where the substrate is integral part of a housing, the thickness may be between 5 mm and 2 cm, and even more. Furthermore, while it is generally preferred that the substrate has a rectangular configuration, other configurations, including round, elliptical, trapezoid, or irregularly shaped are also considered suitable.

Depending on the chemical nature of the substrate, it should be recognized that one or more interposed layers between the substrate and the matrix may be included in the biochip. For example, where the substrate is relatively hydrophobic (e.g., Mylar® 100), a hydrophilic interposed layer may be applied to the substrate where the matrix is relatively hydrophilic. Similarly where the substrate is relatively hydrophilic (e.g., a polycarbonate), a hydrophobic interposed layer may be applied to the substrate where the matrix is relatively hydrophobic. There are numerous materials known in the art that may act as a hydrophilic or hydrophobic interposed layer, and it is generally contemplated that such materials will (a) adhere to at least one surface of the substrate, and (b) improve adhesion of the matrix layer to the substrate (via the interposed layer) when compared to adhesion of the matrix to the substrate without the interposed layer.

For example, particularly suitable hydrophilic interposed layers may include hydrophilic gels and an especially preferred hydrophilic gel comprises gelatin. Alternatively, hydrophilic interposed layers may be formed from organic polymeric compositions (e.g., hydrophilic plastics), or even from heterobifunctional molecules in which one portion has a hydrophobic moiety and another portion has a hydrophilic moiety. Similarly, suitable hydrophobic interposed layers may include silicon-containing materials, and an especially preferred silicon-containing material comprises a silane. Alternatively, hydrophobic interposed layers may be formed from organic polymeric compositions (e.g., hydrophobic plastics, wax), or even from heterobifunctional molecules in which one portion has a hydrophobic moiety and another portion has a hydrophilic moiety.

It is further contemplated that suitable hydrophilic or hydrophobic interposed layers may be applied by various techniques, and a suitable deposition technique will typically and at least in part depend on the particular material employed. For example, where the hydrophilic interposed layer is gelatin, the layer may be poured onto a substrate or a doctor-bladed. Other materials may be deposited by chemical vapor deposition, spin-coating, in situ polymerization, etc. Thus, depending on the desired thickness of the interposed layer, more than one interposed layer may be applied to the substrate wherein each of the layers my have a thickness between 10 microns and even less to about 2 mm, and even more. Alternatively, there are numerous substrates commercially available that already include a hydrophilic or hydrophobic layer (infra). Still further, it should be recognized that where the substrate is hydrophilic and the matrix layer is hydrophilic (or where the substrate is hydrophobic and the matrix layer is hydrophobic), the interposed layer may be entirely omitted.

With respect to the matrix layer, it is preferred that the matrix comprises a meshwork of solubilized or suspended agents, and particularly preferred matrix materials include gelatin, agarose, and polyacrylamide. Suitable alternative matrix materials include porous three-dimensional polymeric networks, semi-interpenetrating or interpenetrating polymeric networks, and so forth. The thickness of contemplated matrix layers may vary substantially, and it is generally contemplated that suitable matrix layers will have a thickness of between 10 microns and 1-10 mm, and even more. However, it is typically preferred that a matrix layer will have a thickness of between 50 microns and 2 mm. Depending on the material of the matrix layer, it should be appreciated that all methods of layer deposition are suitable for use herein, and the same considerations as described above for deposition of the interposed layer apply. Moreover, it should be appreciated that contemplated biochips may include more than one matrix layer, and where more than one matrix layer is employed, it is contemplated that the number of matrix layers is between two and ten (and even higher).

In particularly preferred aspects, the meshwork has a density sufficient to couple/embed and retain a ligand and/or at least part of a coupling moiety. Alternatively, however, other matrix materials are also contemplated suitable so long as such alternative materials will couple/embed and retain a ligand and/or at least part of a coupling moiety.

It is particularly contemplated that the coupling/embedding of the ligand and/or at least part of the coupling moiety may be covalently or non-covalently. For example, where the ligand and/or at least part of the coupling moiety is non-covalently coupled to the matrix, it is contemplated that the matrix material has a density sufficient to retain the ligand and/or at least part of the coupling moiety by steric interaction. Viewed from another perspective, it is contemplated that the meshwork of preferred matrix materials at least partially encloses the ligand and/or at least part of the coupling moiety. On the other hand, where the ligand and/or at least part of the coupling moiety is covalently coupled to the matrix, it is contemplated that the matrix material has at least one reactive group that forms a covalent bond with a reactive group disposed on the ligand and/or at least part of the coupling moiety. There are numerous reactive groups known in the art, and all of those groups are contemplated as being suitable for use herein. For example, where the matrix comprises agarose, suitable groups my include cyanogens bromide or maleic anhydride (which is commercially available as CNBr-agarose or maleic anhydride modified agarose). Alternative reactive groups may include various nucleophilic groups, electrophilic groups, or groups that may specifically react with a crosslinking agent.

Thus, it should be recognized that the ligand in contemplated biochips is coupled to the substrate via a matrix layer (and optionally via an interposed layer), wherein the coupling of the ligand to the matrix may be direct (i.e., without a coupling moiety) or indirect (i.e., with a coupling moiety), and may further be covalent or non-covalent.

In an especially preferred configuration, the ligand is coupled to the matrix layer via a coupling moiety, wherein the coupling moiety has a first portion (e.g., avidin, streptavidin, or modifications thereof) and a second portion (e.g., biotin). In such preferred configurations, it is contemplated that the matrix at least partially embeds or covalently binds the first portion (supra), and that the second portion is covalently bound to the ligand. Due to the highly specific and strong non-covalent binding interaction between the first and second portion of the coupling moiety, the ligand is indirectly coupled to the matrix layer.

Consequently, it should be recognized that in preferred configurations, the matrix layer will comprise a substantially homogenous distribution of the first portion, and that any desired ligand that includes (covalently or non-covalently) the second portion may be coupled to any desired position of the matrix simply by contacting the matrix with the desired ligand, wherein the binding strength of the ligand to the matrix is predominantly determined by the binding strength between the first and second portion of the coupling moiety. Furthermore, by binding the ligand to the matrix via a specific molecular interaction, it should be recognized that no blocking step of the matrix prior to anti-ligand contact is needed. Thus, it should be appreciated that contemplated matrices may be easily and inexpensively customized by a supplier or even end-user.

Of course, it should be recognized that there are numerous alternative first and second portions with high and specific binding to each other to generate a wide variety of coupling partners, and all of such first and second portions are also contemplated suitable herein. For example, a first portion may comprise an antibody or a fragment thereof, while the second portion may comprise the corresponding proteinaceous or non-proteinaceous antigen to the antibody. Alternatively, where a non-proteinaceous molecule is preferred as first and second portion of alternative coupling moieties single-stranded nucleic acids with corresponding complementary sequence may be employed as first and second portions. Still further contemplated first and second portions include those that form stable complexes (i.e., K_{D}≦10⁻⁵M) via one or more specific molecular interactions, including hydrogen bonding, hydrophilic and/or hydrophobic interactions, ionic bonds, electrostatic bonds, etc.

In still further aspects of the inventive subject matter, contemplated matrix layers may include various agents to impart additional functionality to the matrix layer, and particularly preferred functionalities include reduction of at least one of an autofluorescence of the substrate, an incident-light-absorption of the substrate, a charge-effect of the substrate, and a surface unevenness of the substrate. Consequently, it should be appreciated that contemplated biochips may include one or more multi-functional matrix layers.

For example, where the substrate includes a material that exhibits autofluorescence (i.e., has fluorescence without addition of a fluorophore at a particular wavelength), it is contemplated that suitable additional agents in the matrix comprise materials that reduce the autofluorescence. Particularly preferred additional agents comprise mineral oxides (e.g., iron oxide, titanium oxide, etc.), carbon-based materials (e.g., carbon black), and light-absorbing agents with an absorption maximum at the wavelength of the autofluorescence.

Similarly, where the substrate includes a material that absorbs and/or scatters incident light (e.g., incident light to generate fluorescence of a fluorophore coupled to an anti-ligand bound to the ligand), it is contemplated that suitable additional agents in the matrix comprise materials that reduce the absorption and/or scattering of incident light. Particularly preferred additional agents comprise dyes (e.g. direct or indirect dyes, natural or synthetic chromophores), mineral oxides (e.g., iron oxide, titanium oxide, etc.), and carbon-based materials (e.g., carbon black).

In another example, where the matrix comprises a material that exhibits a charge effect, it is contemplated that various agents may be added to the matrix layer that reduce the charge effect (when compared to a matrix not containing the additive). The term “charge effect” as used herein refers to an effect that (a) is at least in part intrinsic to the matrix, and (b) leads to deformation of a droplet (volume between about 100 pl or less to about 50 μl) applied to the matrix from having a circular shape to a non-circular, half-moon, or irregular shape. Particularly preferred additional agents to reduce the charge effect include detergents, wherein such detergents my be ionic, zwitter ionic, and non-ionic detergents. For example, contemplated detergents include Tween®, Brij®, Triton®, various sulfonates and their salts, and various quaternary ammonium compounds and their salts.

In yet another example, it is contemplated that addition of the matrix layer not only provides a layer to which a ligand may be coupled, but it should be recognized that by providing a matrix layer surface unevenness of the substrate may be significantly reduced. Surface unevenness is particularly undesirable in biochips where the detection is based on an optical detection due to the inherent shift in the focal plane when the substrate surface to which the ligand is attached is uneven. FIG. 3 depicts an exemplary biochip 300 with an uneven substrate 310 having an uneven surface 310-S. A first matrix layer 320C is disposed on the uneven surface 310-S, wherein the first matrix layer 320C has a surface 320C-S, with a reduced surface unevenness. A second matrix layer 320B is disposed on the surface 310C-S, wherein the second matrix layer 320B has a surface 320B-S, with a reduced surface unevenness compared to both 320C-S and 310-S. Finally, a third matrix layer 320A is disposed on the surface 310B-S, wherein the third matrix layer 320A has a surface 320A-S, with a reduced surface unevenness compared to 320B-S, 320C-S and 310-S, and wherein the surface 320A-S is substantially planar (maximal vertical deviation between highest and lowest point no more than 10 micron, and more preferably no more than 1 micron). Bound to the ligands (embedded or coupled to the matrix layer) are anti-ligands 340.

In a still further example, it is contemplated that the matrix layer may further comprise a buffer or buffer system that will at least in part provide an appropriate biochemical reaction environment for binding of the anti-ligand to the ligand (e.g., neutral pH and low salt for nucleic acid hybridization), or for a biochemical (enzymatic or non-enzymatic) conversion of the anti-ligand or other component in the biochip or sample fluid (e.g., buffer for luciferase reaction). Suitable buffers and buffer systems may include amphoteric buffers as well as a pair of an acid salt and the corresponding base of the acid. Furthermore, contemplated buffers and buffer systems may include additional reagents to provide a particular chemical environment, and suitable additional reagents include reductants, thiol-reactive agents, salts, preservatives, etc.

In yet another example, it is contemplated that the matrix layer may further comprise one or more humectants to control or reduce the amount of shrinkage and swelling of the matrix. Control over shrinkage and swelling of the matrix is thought to be advantageous in maintaining a particular focal plane, and suitable humectants include glycerol, various oils, sugars, and selected detergents.

It should further be appreciated that while each additional agent may be disposed in a separate matrix layer, combination of one or more additional agents into one layer is generally preferred. For example, a first matrix layer that is coupled to the substrate may include only a humectant, while a second layer may include a buffer, and an agent that reduces the autofluorescence of the substrate. However, the number and distribution of the individual additional agents in one or more of the matrix layers is considered to be not critical to the inventive subject matter.

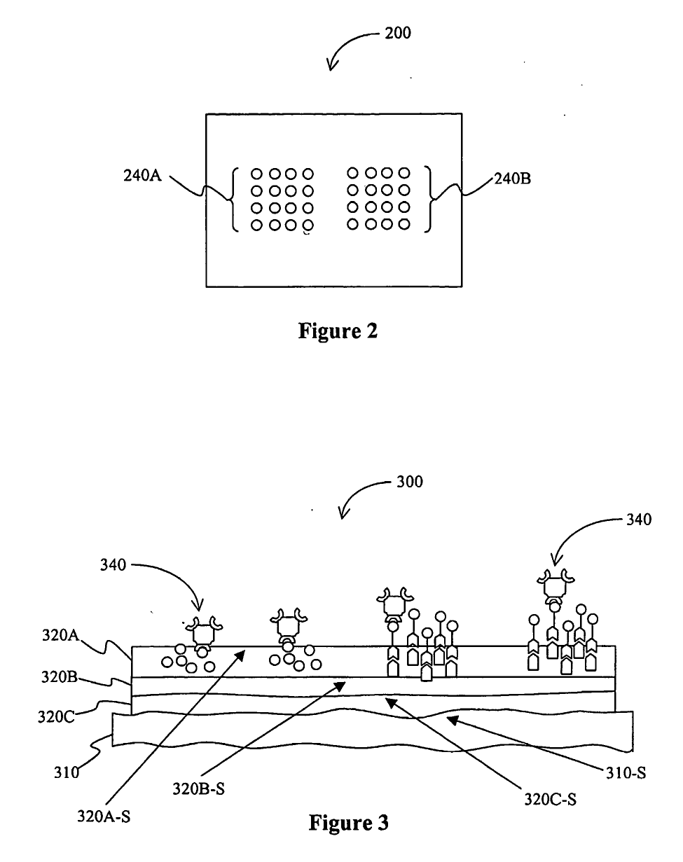

With respect to the ligand, it should be recognized that by virtue of contemplated coupling configurations of the ligand to the matrix layer in present biochips, the particular chemical nature of the ligand is not limiting to the inventive subject matter. Therefore, contemplated ligands include single molecules, complexes of a plurality of molecules, viruses, and even pro-and/or eukaryotic cells or fragments thereof. Examples for particularly preferred molecules include pharmacologically active molecules, nucleosides, nucleotides, oligo- and polynucleotides (single stranded or double stranded), sugars, lipids, amino acids, oligo- and polypeptides (e.g., cytokines, enzymes, antibodies and antibody fragments), hormones, etc. Examples for particularly preferred complexes of a plurality of molecules include multi-component receptors, antibodies (e.g., IgM, IgE, etc.), double and triple stranded nucleic acids, PNA, etc. Examples for particularly preferred virus and virus fragments include DNA and RNA viruses (e.g., HBV, HCV, HIV, RSV, HSV, Influenza, etc.), and examples for pro-and eukaryotic cells include gram-positive bacteria (e.g., Bacillus spec.), gram-negative bacteria (e.g., Escherichia spec.), stem cells, cancer cells, lymphoid cells, immune competent cell, etc. Consequently, it should be especially recognized that contemplated biochips according to the inventive subject matter may include a first plurality of ligands that belong to a first class of molecules (e.g., nucleic acids—oligonucleotide) and include at least a second plurality of ligands that belong to a second class of molecules (e.g., polypeptides-cytokine). An exemplary multi-class biochip 200 is depicted in FIG. 2, in which a first plurality of ligands belonging to a first class of molecules 240A is disposed on a matrix layer proximal to a second plurality of ligands belonging to a second class of molecules 240B.

While it is generally preferred that contemplated biochips include a plurality of chemically distinct ligands, it should also be recognized that where a biochip includes more than one identical ligand, such biochips may be employed for quantification of an anti-ligand by providing different amounts of the ligand to particular positions on the matrix of the biochip.

Thus, the amount of a particular ligand may vary considerably, however, it is generally contemplated that each ligand is present at a particular location in an amount of between about less than 1 pmol to about 1 mmol. Depending on the molecular weight of the ligand, and especially where the ligand is a complex of molecules or a virus or a cell, even lower quantities of the ligand are contemplated and will typically be in the range from a single copy to 1 fmol, or less.

Contemplated ligands may be coupled to the matrix layer in various forms (supra), and it should therefore be appreciated that the manner of application to the matrix layer may vary considerably. However, it is generally preferred that the ligand is applied in a solvent (e.g., buffer) to the surface of the matrix layer, and that the ligand is then retained at or proximal to the surface of the matrix layer via interaction of the first and second portions of the coupling agent. There are numerous methods known in the art to conjugate the second portion to the ligand, and all of the known methods are considered suitable (e.g., biotinylated nucleosides or oligonucleotides are commercially available, biotinylation kits for proteinaceous samples are commercially available, etc.). Alternatively, the ligand may be applied in a solvent (e.g., buffer) to or below the surface of the matrix layer before the matrix layer solidifies, wherein the ligand is then retained at or proximal to the surface by steric interaction with the solidified matrix layer.

Regardless of the amount, chemical nature, and/or method of coupling of the ligand to the matrix layer, it is contemplated that each individual ligand is applied to the matrix layer in a predetermined position, and that binding of an anti-ligand at that position may be detected using numerous methods well known in the art. For example, suitable detection methods include radioisotope detection, optical detection (e.g., fluorescence, luminescence, absorption, etc.), and electrochemical detection.

Consequently, it should be appreciated that the nature of the anti-ligand (i.e. the molecule or material binding to the ligand) will vary considerably, and all molecules or materials are contemplated that form an affinity pair with contemplated ligands. Therefore, contemplated anti-ligands include single molecules, complexes of a plurality of molecules, viruses, and even pro-and/or eukaryotic cells or fragments thereof.

Examples for particularly preferred molecules include pharmacologically active molecules (e.g., antibiotics, antifungals, antiviral agents, enzyme inhibitors, vitamins, antineoplastic agents, DNA- or RNA-binding molecules, etc.), nucleosides, nucleotides, oligo- and polynucleotides (single stranded or double stranded), sugars, lipids, amino acids, oligo- and polypeptides (e.g., cytokines, enzymes, antibodies and antibody fragments), hormones, etc. Examples for particularly preferred complexes of a plurality of molecules include multi-component receptors, antibodies (e.g., IgM, IgE, etc.), double and triple stranded nucleic acids, PNA, etc. Examples for particularly preferred virus and virus fragments include DNA and RNA viruses (e.g., HBV, HCV, HIV, RSV, HSV, Influenza, etc.), and examples for pro-and eukaryotic cells include gram-positive bacteria (e.g., Bacillus spec.), gram-negative bacteria (e.g., Escherichia spec.), stem cells, cancer cells, lymphoid cells, immune competent cell, etc.

Therefore, contemplated sample fluids include all fluids that comprise at least one of the anti-ligands, and especially contemplated sample fluids include biological fluids (e.g., blood, serum, plasma, urine, spinal fluid, sputum, cell lysate, etc.) and non-biological fluids (e.g., buffers, processed biological fluids, etc.), wherein contemplated sample fluids are preferably aqueous fluids. However, where appropriate, it is also contemplated that suitable sample fluids may include non-aqueous fluids, including DMSO, DMF, alcohols, etc.

Consequently, it is contemplated that contemplated biochips comprise a substrate coupled to a multi-functional matrix layer that is coupled to a ligand, wherein the a multi-functional matrix layer provides reduction of at least one of an autofluorescence of the substrate, an incident-light-absorption of the substrate, a charge-effect of the substrate, and a surface unevenness of the substrate, wherein the ligand specifically binds to an anti-ligand that is disposed in a sample fluid when the sample fluid contacts the biochip.

Furthermore, contemplated biochips may comprise a plurality of first ligands in a plurality of first predetermined positions, each of the plurality of first ligands belonging to a class selected from the group consisting of a polypeptide, a polynucleotide, a carbohydrate, a pharmacologically active molecule, a bacterial cell, an eukaryotic cell, and a virus, and a plurality of second ligands in a plurality of second predetermined positions, each of the plurality of second ligands belonging to a class selected from the group consisting of a polypeptide, a polynucleotide, a carbohydrate, a pharmacologically active molecule, a bacterial cell, an eukaryotic cell, and a virus, wherein the class of each of the first ligands and the class of each of the second ligands is not the same. The term “predetermined position” as used herein refers to a predetermined position relative to a reference point on the matrix layer as well as to a predetermined position relative to a position of a ligand on the matrix layer.

Still further, contemplated biochips may comprise a matrix layer coupled to a substrate, wherein the matrix layer has a first surface unevenness and wherein the substrate has a second surface unevenness, wherein the first surface unevenness is less than the second surface unevenness, and a plurality of ligands coupled in predetermined positions to the matrix layer.

In yet further aspects, contemplated biochips may comprise a substrate at least partially covered with a matrix layer, wherein a ligand is applied to the matrix layer in a liquid, wherein the ligand binds to the matrix layer from the liquid, and wherein the matrix layer has a reduced charge-effect sufficient to allow formation of a substantially circular ligand deposition area when the ligand is applied to the matrix layer.

In yet further aspects, contemplated biochips comprise a substrate at least partially coupled to a matrix layer, wherein the matrix layer is further coupled to a plurality of ligands in a plurality of predetermined positions, and wherein at least one of the plurality of ligands binds to an anti-ligand that is disposed in a sample fluid when the sample fluid contacts the biochip.

### EXAMPLES

The following examples are provided to illustrate manufacture of an exemplary biochip according to the inventive subject matter. However, it should be recognized that numerous modifications may be made without departing from the inventive concept presented herein.

### Experiment 1—Matrix Coating and Fluorophore Detection

Optically pure, 100 micron thick Mylar with a gelatin coating was obtained from the Dupont Corporation (Dupont Corp., Cat. No, P4CIA). A solution of agarose in water was prepared as described below. Cy3 marker (NEN Life Sciences) was added to the solution and mixed thoroughly yielding a uniform suspension. The Cy3-agarose solution was then spread evenly over the carrier using a Leneta Wire-Cator (BYK-Gardner Corporation) as described below. The coating was allowed to cool. Using a Bio-Rad MRC-1024 Confocal Microscope and Omnichrome 643 100 Kr—Ar laser (Bio-Rad Laboratories, Hercules, Calif.), multiple 200 micron areas of the coated carrier were successively excited with a wavelength of 550nm. The microscope detected an image over every 200 micron area of the surface of the matrix using a detection (emission) wavelength of 570 nm. This experiment demonstrated that an aqueous matrix adheres to a hydrophobic polymeric substrate in which one side has been rendered hydrophilic by a hydrophilic interposed layer. Furthermore, this experiment demonstrates that the hydrophilic interposed matrix layer does not interfere with fluorescence detection of a fluorescent marker.

### Experiment 2—Concentration of Light Blocking Agent

A 2% solution of agarose in water and 6 gm of iron oxide as a light-blocking agent was prepared as described below. The Cy3-agarose coated carrier from Experiment 1 was coated onto the substrate from Experiment 1 to make a 200 micron layer of the iron oxide-agarose solution and allowed to cool. Using the same procedure from Experiment 1, an image was detected over every 200 micron area of the surface of the matrix. The iron oxide-agarose coating step was repeated five times on the same substrate until no image was detected on the surface of the matrix using the confocal microscope of Experiment 1. The total concentration of iron oxide that completely blocked the Cy3 image identified the amount of light blocking agent needed to render the carrier optically inactive (i.e., to suppress autofluorescence from the substrate or absorption of incident light of the substrate). It should be understood that one of skill in the art can determine the amount of any light blocking agent required to render a carrier optically inactive using the above procedure. Thus, this experiment demonstrates how to determine the amount of an agent that reduces autofluorescence or incident light absorption required to render the carrier optically inactive.

### Experiment 3—Single Matrix Layer Configuration

The procedure of Experiment 2 was duplicated using a single layer of iron oxide-agarose solution, at the concentration identified in Experiment 2. Again, using the confocal microscope and laser of Experiment 1, the Cy3 image was completely blocked. This experiment demonstrates that the total amount of light blocking agent required to render the carrier optically inactive can be applied in a single coating.

### Experiment 4—Coupling of a Ligand to a Matrix

A 2 wt % solution of agarose in water containing 2.25 wt % streptavidin was prepared as described below. The streptavidin agarose solution was then coated onto one surface of the substrate from Experiment 1. An aliquot of Oligo A (NEN Life Science Products) was then deposited in aqueous buffer onto the surface of the entire matrix and allowed to cross-link with the matrix via biotin-straptavidin interaction. Using the confocal microscope and laser of Experiment 1, an image was detected over every 200 micron area of the surface of the matrix. This experiment demonstrated that ligands can be coupled to the a matrix using a coupling moiety in which one portion is coupled to the ligand and in which another portion is coupled (here: embedded) in the matrix.

Oligo A—SEQ ID: 1, biotinylated at 5′ position: Cy3 labeled at 3′ position.

SEQ ID: 1 =
5′-AAT CCA GAT ATA GTC ATC TAG CAA TAC A-3′

### Experiment 5—Adhesion of Ligand to the Matrix

The procedure of Experiment 4 was repeated. However, the coated substrate was repeatedly washed with deionized water following the addition of Oligo A to verify that the sensing element was bound essentially irreversibly to the surface of the matrix. Using the confocal microscope and laser of Experiment 1, an image was detected over every 200 micron area of the surface of the matrix. This experiment demonstrated coupling of a ligand to a matrix via a coupling moiety is not affected by subsequent washes with deionized water.

### Experiment 6—Light Blocking Agent

The solution of agarose in water with streptavidin from Experiment 4 was coated on one side of the substrate of Experiment 1. Then the solution of light blocking agent in agarose from Experiment 3 was coated over the initial matrix coating. Using the confocal microscope and laser of Experiment 1, no image was detected. This experiment demonstrated that the light blocking agent disposed over the matrix containing a cross-linking system renders the carrier optically inactive.

### Experiment 7—Light blocking agent and Coupling Moiety containing Matrix Layer

A solution of aldehyde activated agarose, commercially available as NuFIX™, was prepared with strepavidin, as described below. The procedure of Experiment 6 was repeated. The NuFIX™ solution was then coated over the matrix layer containing light-blocking agent and allowed to cool. Using the confocal microscope and laser of Experiment 1, no image was detected. This experiment demonstrated that a matrix layer containing crosslinking agents, disposed over a matrix layer containing the light blocking agent has no affect on the light blocking agent's ability to render the carrier optically inactive.

### Experiment 8—Labeled Ligand Cross-Linked to a Matrix Layer

The procedure of Experiment 7 was repeated. Then an aliquot of Oligo A from Experiment 4 was deposited over the surface of the matrix layer and allowed to cross-link. Using the confocal microscope and laser of Experiment 1, an image was detected over every 200 micron area of the surface of the matrix. This experiment demonstrated that a fluorescence-labeled ligand produces a detectable fluorescence signal when bound to the top surface of matrix layers that include a light-blocking agent.

### Experiment 9—Unlabeled Ligand Produces No Image

The procedure of Experiment 7 was followed using Oligo B (NEN Life Science Products). Using the confocal microscope and laser of Experiment 1, no image was detected. This experiment demonstrates that an unlabeled ligand bound to the surface of a matrix layer does not produce a colormetric signal.

Oligo B—SEQ ID: 1, biotinylated at 5′ position: not labeled at 3′ position.

SEQ ID: 1 =
5′-AAT CCA GAT ATA GTC ATC TAG CAA TAG A-3′

### Experiment 10—Detection of an Anti-ligand bound to a Ligand bound a Matrix Layer

The procedure of Experiment 9 was repeated. An aliquot of Oligo C (NEN Life Science Products), which is partially complementary to Oligo B, was deposited over the surface of the matrix and allowed to hybridize with Oligo B. This experiment was performed in triplicate, allowing Oligo C to hybridize for 4 hours, 8 hours, and overnight. Using the confocal microscope and laser of Experiment 1, images were detected over every 200 micron area of the surfaces of each of the matrices. This experiment demonstrated that a fluorescently-labeled anti-ligand (e.g., nucleic acid) that hybridizes to a Ligand bound to the surface of the matrix produces a fluorescence signal.

Oligo B—SEQ ID: 1, biotinylated at 5′ position: not labeled at 3′ position.

SEQ ID: 1 =
5′-AAT CCA GAT ATA GTC ATC TAG CAA TAC A-3′
SEQ ID: 2 =
5′-TTA GCT CGA CTC AGG GAT CCG GAT TGT ATT GCT
AGA-3′

### Experiment 11—Formation of a Biochip

A 210 micron pin was obtained from TeleChem International, Inc., Sunnyvale, Calif. A corresponding 4-spot metal spotting block was machined at a local metal fabricating shop such that the weight of the pin spotter determines the amount of oligo that is deposited. The procedure of Experiment 7. was repeated. Two spots of Oligo B from Experiment 9 were then spotted onto the surface of the matrix. Using the confocal microscope and laser of Experiment 1, no image was detected. The device was then washed with deionized water and placed in the confocal microscope for another reading. No image was detected. This experiment demonstrated that unlabeled nucleic acid ligands spotted onto the surface of the matrix do not produce a fluorescence signal.

### Experiment 12—Hybridization Assay on a Biochip

The procedure of Experiment 11 was repeated. An aliquot of Oligo C (NEN Life Science Products) was then deposited over the surface of the matrix and allowed to hybridize with Oligo B, which was spotted onto the surface of the matrix. After washing with deionized water, two distinct spot images were detected on the surface of the matrix using the confocal microscope and laser of Experiment 1. Finally, stray light was allowed to contact the bottom surface of the matrix during another reading with the confocal microscope. The stray light did not produce an adverse effect on the images detected.

This experiment demonstrated that the ligands bound to the surface of the matrix remain fixed in position and do not migrate over the surface of the matrix during the procedure. Furthermore, this experiment demonstrated that stray light impinging upon the device from surfaces other than the top did not affect the light blocking agent's ability to render the carrier optically inactive.

### Experiment 13—Nucleic Acid Sandwich Hybridization Assay Performed on the Biochip

A standard nucleic acid sandwich hybridization assay was carried out on the present biochip. The procedure of Experiment 11 was repeated. An aliquot of Oligo E (NEN Life Science Products) was deposited over the surface of the matrix and allowed to hybridize with Oligo B, which was spotted onto the surface of the matrix. After washing with deionized water, an aliquot of Oligo F was deposited over the surface of the matrix and allowed to hybridize with Oligo E. After washing with deionized water, two distinct spot images were detected on the surface of the matrix using the confocal microscope and laser of Experiment 1. One of ordinary skill in the art would refer to Sambrook, J. et al. vol. 1-3, Molecular Cloning, A Laboratory Manual, 1989 for a detailed description of nucleic acid sandwich hybridization assays.

Oligo B—SEQ ID: 1, biotinylated at 5′ position: not labeled at 3′ position.

Oligo E—SEQ ID: 2, not labeled at 3′ position.

Oligo F—SEQ ID: 3, Cy3 labeled at 3′ position.

SEQ ID: 1 =
5′-AAT CCA GAT ATA GTC ATC TAG CAA TAC A-3′
SEQ ID: 2 =
5′-TTA GCT CGA CTC AGG GAT CCG GAT TGT ATT GCT
AGA-3′
SEQ ID: 3 =
5′-ATC CGG ATC CCT-3′

### Experiment 14—Sandwich Immunoassay for IL-2

A commercially available capture antibody (e.g., anti-IL-2) was coupled to biotin using protocols well known in the art, and the biotinylated antibody was printed on a streptavidin-coated matrix using a PixSys 3500 dispensing workstation (Cartesian Technologies, CA). The antibody spots were incubated on the matrix for 15 minutes at room and washed three times using 0.1 M Tris buffer (pH 7.6) containing 0.1% Tween-20 and 0.01% sodium azide. 100 μl of interleukin-2 at 10 ng/ml was dispensed into the microarray and the assay was incubated for 45 minutes at room temperature. Following IL-2 incubation, 100 μl of a Cy-5 labeled antibody (anti-IL-2) solution was added and the assay was incubated for 45 minutes at room temperature. The microarray was washed three times using 0.1 M Tris buffer (pH 7.6) containing 0.1% Tween-20 and 0.01% sodium azide. The spots were dried using compressed air and scanned at 10 micron resolution using ScanArray 5000 XL (Packard BioScience, MA). All spots exhibited substantially identical fluorescence intensity.

### Depositing or Coating of a Matrix

An exemplary method for depositing a matrix layer onto the substrate involves quickly dispensing a melted material, suitable for use as a matrix coating, onto the carrier. Just after the solution spreads over the surface of the substrate, a Leneta Wire-Cator or a Film applicator capable of delivering a prescribed volume per square meter is drawn down the carrier to spread liquefied matrix material evenly over the surface of the substrate. Such coating applicators are commercially available from BYKGardner Corporation. The coated substrate is kept at room temperature until the matrix layer was dry.

### First Matrix Layer

Heat about 1.5 liters of water in a beaker until approximately 60° C. and pour into a 1 liter graduated cylinder. Place a wire wound coating rod capable of delivering a 50 ml per square meter coverage into the cylinder in order to warm it. Cut a piece of 5.25 inch gelatin-coated mylar (polyester) carrier about 20 inches long and tape one short edge onto a flat surface such as a lab bench. Ensure that the hydrophilic side (gelatin coated side) is facing up. Use a spatula and measure 10 grams of 2% agarose into a conical centrifuge/culture tube. Add 10 ml of Buffer A. Place the loosely capped tube into a microwave and heat on high in 20 second increments until melted. Be careful not to boil over the tube. Working quickly, remove the wire wound rod from the warm water and dry with a paper towel. Place at the top of the substrate. Remove the culture tube from the water bath and pour about ½ of the contents over the coating rod. Just after solution spreads onto the substrate, lightly grab the rod and draw down the coating. Allow the coated substrate to sit at room temperature for 20 minutes. It can then be allowed to air dry for at least 2 hours.

### Second Matrix Layer

Heat about 1.5 liters of water in a beaker until approximately 60° C. and pour into a 1 liter graduated cylinder. Place a wire wound coating rod capable of delivering a 200 ml per square meter coverage into the cylinder in order to warm it. Tape down the substrate upon which the first matrix level has been deposited or coated, coating side up, onto a flat surface such as a lab bench. Use a spatula and measure 10 grams of 2% agarose with iron oxide into a conical centrifuge/culture tube. Add 10 ml of Buffer B. Place the loosely capped tube into a microwave and heat on high in 20 second increments until melted. Be careful not to boil over the tube. Working quickly, remove the wire wound rod from the warm water and dry with a paper towel. Place at the top of the coated carrier. Remove the culture tube from the water bath and pour about ½ of the contents over the coating rod. Just after solution spreads onto the coated substrate, lightly grab the rod and draw down the coating. Allow the coated substrate to sit at room temperature for 20 minutes. It can then be allowed to air dry for at least 2 hours.

### Third Matrix Layer

Measure 20 grams of NuFIX™ and place into a 2 liter beaker. Add 1 liter of deionized water and place on a hot plate to slowly bring to a boil. Stir occasionally with a glass rod until all of the agarose is completely melted. Turn off heat and allow mixture to cool to room temperature with an aluminum foil cover over the beaker to keep out dust. After hardening, the agarose should be labeled and can be tightly sealed and stored in a refrigerator for later use.

Use a spatula and measure 10 grams of 2% NuFIX™ into a conical centrifuge/culture tube. Add 2 ml of Buffer C. Place the loosely capped tube into a microwave and heat on high in 20 second increments until melted. Be careful not to boil over the tube. In a 10 ml disposable culture tube, prepare 3.0 ml of a 10 mg/ml solution of Streptavidin in Buffer C. Warm the Streptavidin solution to 50° C. in a water bath. After the Streptavidin solution has warmed, remove 2.5 ml and add it to the tube from step 4 above. Mix the 14.5 ml NuFIX™/Streptavidin solution and incubate for 2 hours in a 50° C. water bath. While the NuFIX™/Streptavidin solution reacts, prepare the following: In a 10 ml disposable conical tube, add 1 ml of 1.33×1 sodium cyanoborohydride to 1 ml Buffer D solution; In a second 10 ml disposable conical tube, add 1.5 ml of 300 ml histidine to 20 ml Buffer D solution. After the 2 hour incubation of the NuFIX™/Streptavidin solution, add 1.67 ml of 300 mM histidine. Mix thoroughly and incubate at 50° C. for 1 hour. After 1 hour of incubation, add 0.29 ml of 1.33 M sodium cyanoborohydride. Add 0.04 gm Tween-20 and mix thoroughly and react for 1 hour at 50° C. Do not cap the tube tightly as hydrogen gas may be produced. Allow tube to vent. Heat about 1.5 liters of water in a beaker until approximately 60° C. and pour into a 1 liter graduated cylinder. Place a wire wound coating rod capable of delivering a 200 ml per square meter coverage into the cylinder in order to warm it. Tape down the coated substrate, coating side up, onto a flat surface such as a lab bench. Working quickly, remove the wire wound rod from the warm water and dry with a paper towel. Place at the top of the coated substrate. Remove the culture tube from the water bath and pour about ½ of the contents over the coating rod. Just after solution spreads to onto the carrier, lightly grab the rod and draw down the coating. Allow the coated stage to sit at room temperature for 20 minutes. It can then be allowed to air dry for at least 2 hours.

### Preparation of Affinity Matrix with a Plurality of Ligands

Commonly used techniques in the art are available for depositing ligands onto the surface of the platform. The ligands are typically deposited in droplets with a spot diameter ranging from about 20 to 1000 μm, preferably 120-200 μlm spaced apart. Contemplated matrices are suitable for spotting by all contact and non-contact methods. Spotting devices and techniques known in the art include, but are not limited to, syringe-solenoid, solid pin replicator, quill and split pin, tweezer, Pin-and-Ring™, and jetting and piezoelectric pumps. One of ordinary skill in the art would refer to “Microarray Biochip Technology” Ed. Mark Schena, Eaton Publishing, Natick, Mass., 2000, for a detailed description of spotting techniques.

Incubation of the anti-ligand(s) with the ligands on contemplated biochips is generally performed in a period between several seconds to several hours at conditions that allow specific binding of the anti-ligand to the ligand. After incubation, the biochip may be washed with wash agents that may or may not be buffered, or contain detergents.

2% Agarose (e.g., for first matrix layer): Measure 20 grams of dry agarose and place into 2 liter beaker. Add 1 liter of deionized water and place on hot plate to slowly bring to a boil. Stir occasionally with a glass stir rod until all of the agarose is completely melted. Turn off heat and allow mixture to cool to room temperature with aluminum foil cover over beaker to keep out dust. After hardening, the agarose should be labeled and can be tightly sealed and stored in a refrigerator for later use.

2% Agarose with iron oxide (Light Blocking Agent) (e.g., for second matrix layer): Measure 20 grams of dry agarose and place into 2 liter beaker. Add 500 ml of deionized water and place on a hot plate to slowly bring to a boil. Stir occasionally with a glass rod until all of the agarose is completely melted. Measure 6 grams of iron oxide into container and mix until completely and homogeneously dispersed. In a second 2 liter beaker, warm about 750 ml of deionized water to about 80° C. Bring the volume of the iron oxide perparation up to 1 liter by adding 80° C. deionized water. Mix the dispersion until smooth and completely dispersed. Turn off heat and allow mixture to cool to room temperature with an aluminum foil cover over the beaker to keep out dust. After hardening, the agarose should be labeled and can be tightly sealed and stored in a refrigerator for later use.

Buffer A: Add 100 ml of deionized water to a 250 ml Erlenmyer flask with a small stir bar. Place on a magnetic stirrer and stir slowly. Add 0.01 moles of Hepes free acid and allow to dissolve. Measure pH and adjust to 7.4±0.05 using either 0.5 molar NaOH or 0.5 molar HCl. Add 0.02 grams of Tween-20. Bring volume in the flask up to 200 ml using deionized water and stir to dissolve contents completely.

Buffer B: Add 100 ml of deionized water to a 250 ml Erlenmver flask with a small stir bar. Place on a magnetic stirrer and stir slowly. Add 0.01 moles of methane-ethane sulphonic acid (MES) and allow to dissolve completely. Measure pH and adjust to 6.8±0.05 using either 0.5 molar NaOH or 0.5 molar HCl. Add 0.03 grams of Tween-20. Bring volume in the flask up to 200 ml using deionized water and stir to dissolve contents completely.

Buffer C: Add 100 ml of deionized water to a 250 ml Erlenmeyer flask with a small stir bar, place on a magnetic stirrer and stir slowly. Add 1.0 mM of dibasic sodium phosphate. Measure pH and adjust to 5.8±0.05 using either 0.5 molar NaOH or 0.5 molar HCl. Bring volume in the flask up to 200 ml using deionized water and stir to dissolve contents completely.

Buffer D: Add 100 ml of deionized water to a 250 ml Erlenmyer flask with a small stir bar, and place on a magnetic stirrer and stir slowly. Add 2.0 mM of dibasic sodium phosphate. Measure pH and adjust to 7.0±0.05 using either 0.5 molar NaOH or 0.5 molar HCl. Bring volume in the flask up to 200 ml using deionized water and stir to dissolve contents completely.

Thus, specific embodiments and applications of improved biochips have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A biochip comprising:
a substrate coupled to a multi-functional matrix layer that is coupled to a ligand, wherein the a multi-functional matrix layer provides reduction of at least one of an autofluorescence of the substrate, an incident-light-absorption of the substrate, a charge-effect of the substrate, and a surface unevenness of the substrate; and
wherein the ligand binds to an anti-ligand that is disposed in a sample fluid when the sample fluid contacts the biochip.

2. The biochip of claim 1 wherein the substrate comprises an organic polymer or an inorganic polymer.

3. The biochip of claim 2 wherein the polymer is selected from the group consisting of a polyethylene, a polyester, and a polystyrene.

4. The biochip of claim 1 further comprising a hydrophilic interposed layer between the substrate and the multi-functional matrix layer.

5. The biochip of claim 1 wherein the multi-functional matrix layer comprises an aqueous solvent.

6. The biochip of claim 5 wherein the multi-functional matrix layer comprises a material selected from the group consisting of an agarose, a polyacrylamide, and a gelatin.

7. The biochip of claim 1 further comprising a second multi-functional matrix layer wherein the second multi-functional matrix layer is coupled to the multi-functional matrix layer and provides reduction of at least one of an autofluorescence of the substrate, an incident-light-absorption of the substrate, a charge-effect of the substrate, and a surface unevenness of the substrate.

8. The biochip of claim 1 wherein the ligand comprises at least one of a nucleotide, a polypeptide, a polynucleotide, a carbohydrate, and a pharmacologically active molecule.

9. The biochip of claim 1 wherein the ligand is at least partially embedded within the multi-functional matrix layer.

10. The biochip of claim 1 wherein the ligand is coupled to the multi-functional matrix layer via a coupling moiety.

11. The biochip of claim 10 wherein the coupling moiety comprises a first portion that is coupled to the matrix layer and a second portion that is coupled to the ligand, and wherein the first and second portions form a non-covalent bond with each other.

12. The biochip of claim 11 wherein the first portion comprises avidin or streptavidin, and wherein the second portion comprises biotin.

13. The biochip of claim 1 wherein the anti-ligand is selected from the group consisting of a polypeptide, a polynucleotide, a carbohydrate, and a pharmacologically active molecule.

14. The biochip of claim 13 wherein the sample fluid comprises a fluid selected from the group consisting of blood, serum, plasma, urine, spinal fluid, sputum, cell lysate, and a buffer.

15. A biochip comprising:
a plurality of first ligands in a plurality of first predetermined positions, each of the plurality of first ligands belonging to a class selected from the group consisting of a polypeptide, a polynucleotide, a carbohydrate, and a pharmacologically active molecule;
a plurality of second ligands in a plurality of second predetermined positions, each of the plurality of second ligands belonging to a class selected from the group consisting of a polypeptide, a polynucleotide, a carbohydrate, and a pharmacologically active molecule; and
wherein the class of each of the first ligands and the class of each of the second ligands is not the same.

16. The biochip of claim 15 wherein the plurality of first and second ligands are coupled to a matrix layer that is coupled to a substrate.

17. The biochip of claim 16 wherein the class of the plurality of first ligands is an oligonucleotide and the class of the plurality of second ligand is an antibody or a cytokine.

18. The biochip of claim 16 further comprising a second matrix layer and wherein at least one of the matrix layer and the second matrix layer is a multi-functional matrix layer that provides reduction of at least one of an autofluorescence of the substrate, an incident-light-absorption of the substrate, a charge-effect of the substrate, and a surface unevenness of the substrate.

19. The biochip of claim 16 wherein the matrix layer comprises an aqueous solvent and further comprises a material selected from the group consisting of an agarose, a polyacrylamide, and a gelatin.

20. A biochip comprising:
a matrix layer coupled to a substrate, wherein the matrix layer has a first surface unevenness and wherein the substrate has a second surface unevenness, wherein the first surface unevenness is less than the second surface unevenness; and
a plurality of ligands coupled in predetermined positions to the matrix layer.

21. The biochip of claim 20 wherein the substrate comprises an organic polymer or an inorganic polymer, and wherein the matrix layer comprises an aqueous solvent and a material selected from the group consisting of an agarose, a polyacrylamide, and a gelatin.

22. The biochip of claim 20 wherein at least one of the plurality of ligands comprises one of a nucleotide, a polypeptide, a polynucleotide, a carbohydrate, and a pharmacologically active molecule.

23. The biochip of claim 20 wherein at least one of the plurality of ligands is at least partially embedded within the matrix layer.

24. The biochip of claim 20 wherein at least one of the plurality of ligands is coupled to the matrix layer via a coupling moiety.

25. The biochip of claim 24 wherein the coupling moiety comprises a first portion that is coupled to the matrix layer and a second portion that is coupled to the ligand, and wherein the first and second portions form a non-covalent bond with each other.

26. A biochip comprising:
a substrate at least partially covered with a matrix layer, wherein a ligand is applied to the matrix layer in a liquid;
wherein the ligand binds to the matrix layer from the liquid; and
wherein the matrix layer has a reduced charge-effect sufficient to allow formation of a substantially circular ligand deposition area when the ligand is applied to the matrix layer.

27. The biochip of claim 26 wherein the substrate comprises an organic polymer or an inorganic polymer.

28. The biochip of claim 26 wherein the matrix layer comprises an aqueous solvent and further comprises a material selected from the group consisting of an agarose, a polyacrylamide, and a gelatin.

29. The biochip of claim 28 wherein the matrix layer further comprises a material selected from the group consisting of a surfactant, a humectant, a buffer, and a light-absorbing agent.

30. The biochip of claim 26 further comprising a hydrophilic interposed layer between the substrate and the matrix layer.

31. The biochip of claim 26 further comprising at least a second matrix layer wherein the second matrix layer is coupled to the matrix layer.

32. The biochip of claim 26 wherein binding of the ligand includes a coupling moiety.

33. The biochip of claim 32 wherein the coupling moiety comprises a first portion that is coupled to the matrix layer and a second portion that is coupled to the ligand, and wherein the first and second portions form a non-covalent bond with each other.

34. A biochip comprising a substrate at least partially coupled to a matrix layer, wherein the matrix layer is further coupled to a plurality of ligands in a plurality of predetermined positions, and wherein at least one of the plurality of ligands binds to an anti-ligand that is disposed in a sample fluid when the sample fluid contacts the biochip.

35. The biochip of claim 34 wherein the substrate comprises an organic polymer or an inorganic polymer.

36. The biochip of claim 34 further comprising a hydrophilic interposed layer between the substrate and the matrix layer.

37. The biochip of claim 34 wherein the matrix layer comprises an aqueous solvent and further comprises a material selected from the group consisting of an agarose, a polyacrylamide, and a gelatin.

38. The biochip of claim 34 further comprising at least a second matrix layer coupled to the matrix layer.

39. The biochip of claim 34 wherein at least one of the plurality of ligands comprises at least one of a nucleotide, a polypeptide, a polynucleotide, a carbohydrate, and a pharmacologically active molecule.
